(19) United States
(12) Patent Application Publication
(10) Pub. No.: US 2023/0012206 A1
(43) Pub. Date: Jan. 12, 2023

(54) SYSTEMS AND METHODS FOR MULTI-LEVEL THERMAL MANAGEMENT OF ELECTRONIC DEVICES

(71) Applicants: Baxter International Inc., Deerfield, IL (US); Baxter Healthcare SA, Glattpark (Opfikon) (CH)

(72) Inventors: Jiri Slaby, Buffalo Grove, IL (US); Matthew Stephen Vogel, Spring Grove, IL (US); Ford Christopher Ellis, Kenosha, WI (US)

(21) Appl. No.: 17/863,036

(22) Filed: Jul. 12, 2022

Related U.S. Application Data
(60) Provisional application No. 63/220,720, filed on Jul. 12, 2021.

(51) Int. Cl. H02J 7/00; G01K 13/00

(52) U.S. Cl. CPC H02J 7/007194; G01K 13/00

(57) ABSTRACT

The present disclosure provides a new and innovative method and system for the thermal management of devices, such as medical devices and other electronic devices. In various embodiments, a computer-implemented method includes measuring a current temperature of a device drawing power at a charge current level, identifying a current temperature threshold in a plurality of temperature thresholds based on the current temperature, the plurality of temperature thresholds including at least one rising temperature threshold and at least one falling temperature threshold, increasing the charge current level of the device when the current temperature is below a falling temperature threshold for a first period of time, and decreasing the charge current level of the device when the current temperature is above the rising temperature threshold for the first period of time.

### CROSS REFERENCE TO RELATED APPLICATIONS

The instant application claims priority to U.S. Provisional Patent Application No. 63/220,720, entitled “Systems and Methods for Multi-Level Thermal Management of Electronic Devices” and filed Jul. 12, 2021, the disclosure of which is hereby incorporated by reference in its entirety.

### TECHNICAL FIELD

The instant application is directed towards electronic devices and more specifically to control systems controlling the performance of electronic devices.

### BACKGROUND

The present disclosure provides a new and innovative method and system for the thermal management of devices, including medical devices. In various embodiments, the device is an infusion pump. Generally, medical patients sometimes require precise delivery of either continuous medication or medication at set periodic intervals. Medical pumps have been developed to provide controlled drug infusion wherein the drug can be administered at a precise rate that keeps the drug concentration within a therapeutic margin and out of an unnecessary or possibly toxic range. The medical pumps can provide appropriate drug delivery to the patient at a controllable rate, which does not require frequent attention.

Medical pumps may facilitate administration of intravenous therapy to patients both in and outside of a clinical setting. Outside a clinical setting, doctors have found that in many instances patients can return to substantially normal lives, provided they receive periodic or continuous intravenous administration of medication. Among the types of therapies requiring this kind of administration are antibiotic therapy, chemotherapy, pain control therapy, nutritional therapy, and several other types known by those skilled in the art. In many cases, patients receive multiple daily therapies. Certain medical conditions require infusion of drugs in solution over relatively short periods such as from thirty minutes to two hours. These conditions and others have combined to promote the development of increasingly lightweight, portable, or ambulatory infusion pumps that can be worn by a patient and are capable of administering a continuous supply of medication at a desired rate, or providing several doses of medication at scheduled intervals.

Configurations of infusion pumps include elastomeric pumps, which squeeze solution from flexible containers, such as balloons, into IV tubing for delivery to the patient. Alternatively, spring-loaded pumps pressurize the solution containers or reservoirs. Certain pump designs utilize cartridges containing flexible compartments that are squeezed by pressure rollers for discharging the solutions. Infusion pumps utilizing syringes are also known wherein a drive mechanism moves a plunger of the syringe to deliver fluid to a patient. Typically, these infusion pumps include a housing adapted to receive a syringe assembly, a drive mechanism adapted to move the syringe plunger, a pump control unit having a variety of operating controls, and a power source for powering the pump including the drive mechanism and controls.

Additionally, some infusion pumps are portable, for example, an infusion pump may be smaller and more compact for mobile use by ambulatory patients or other patients. Naturally, a portable pump must be supplied with an equally portable power source as a means for powering the pump motor. Batteries are a suitable choice of power for portable units. Some pumps may use disposable batteries while other pumps may use rechargeable batteries. The pump may also be sized to be attached to an I.V. pole. The I.V. pole, with attached pump, may remain stationary or may be moved about in a hospital setting. In another example, the pump may be attached to a hospital bed or other support structure. As noted above, the pump may be portable and may be carried on the patient, for example, in a pouch. The pump may be attached to and supported by the patient's clothing and/or other support apparel such as a belt, a vest, or the like.

Rechargeable batteries are widely used as a power source for various types of systems, such as infusion pumps. The longevity of a battery stored in a system is a critical factor to the performance of that system. However, rechargeable battery longevity is reduced when the battery pack is overheated due to excessive current being provided to the battery pack. Mitigating the damage to the battery pack can be achieved by ensuring the temperature of the battery pack does not exceed a threshold temperature.

Several methods exist to ensure batteries stored in systems are not damaged by excessive temperature, thereby extending the lifetime of the battery. However, the existing methods have several disadvantages. For example, one existing method for ensuring a battery pack stored in a system is not damaged by excessive temperature is to utilize fans or other active cooling devices to manage the thermal performance of a device. These methods, however, have a variety of limitations and drawbacks. For example, active cooling devices increase the overall cost of the device and increase power demand for the device as the active cooling devices require power. This can be particularly problematic for battery-powered devices that already have a fixed amount of power for the device. Active cooling devices may also have life limitations that are shorter than the overall life of the medical device, requiring repair and/or premature replacement of the medical device.

Accordingly, a method and system for managing the thermal performance of a rechargeable battery in a system that does not require user intervention is desired.

### SUMMARY

The present disclosure provides a new and innovative method and system for the thermal management of devices, such as medical devices and other electronic devices. In various embodiments, a computer-implemented method includes measuring a current temperature of a device drawing power at a charge current level, identifying a current temperature threshold in a plurality of temperature thresholds based on the current temperature, the plurality of temperature thresholds including at least one rising temperature threshold and at least one falling temperature threshold, increasing the charge current level of the device when the current temperature is below a falling temperature threshold for a first period of time, and decreasing the charge current level of the device when the current temperature is above the rising temperature threshold for the first period of time.

In various embodiments, the computer-implemented method of claim 1 further includes determining the current temperature is above the rising temperature threshold for a second period of time and setting the charge current level of the device to a lowest charge current level.

In various embodiments, the computer-implemented method of claim 1 further includes determining when the current temperature is above an alert threshold and immediately setting the charge current level of the device to a lowest charge current level.

In various embodiments, the computer-implemented method of claim 1 further includes determining the current temperature is below the falling temperature threshold for a second period of time and increasing the charge current level by one level.

In various embodiments, the power is used to charge a battery.

In various embodiments, the current temperature includes a battery temperature.

In various embodiments, the current temperature includes a processor temperature.

In various embodiments, a device includes a processor, at least one temperature sensor, and a memory storing instructions that, when read by the processor, cause the device to measure, using the at least one temperature sensor, a current temperature of the device drawing power at a charge current level, identify a current temperature threshold in a plurality of temperature thresholds based on the current temperature, the plurality of temperature thresholds including at least one rising temperature threshold and at least one falling temperature threshold, increase the charge current level of the device when the current temperature is below a falling temperature threshold for a first period of time, and decrease the charge current level of the device when the current temperature is above the rising temperature threshold for the first period of time.

In various embodiments, the instructions, when read by the processor, further cause the device to determine the current temperature is above the rising temperature threshold for a second period of time and set the charge current level of the device to a lowest charge current level.

In various embodiments, the instructions, when read by the processor, further cause the device to determine when the current temperature is above an alert threshold and immediately set the charge current level of the device to a lowest charge current level.

In various embodiments, the instructions, when read by the processor, further cause the device to determine the current temperature is below the falling temperature threshold for a second period of time and increase the charge current level by one level.

In various embodiments, the power is used to charge a battery.

In various embodiments, the current temperature includes a battery temperature.

In various embodiments, the current temperature includes a processor temperature.

In various embodiments, a non-transitory computer readable medium storing instructions that, when executed by one or more processors, cause the one or more processors to perform steps including measuring a current temperature of a device drawing power at a charge current level, identifying a current temperature threshold in a plurality of temperature thresholds based on the current temperature, the plurality of temperature thresholds including at least one rising temperature threshold and at least one falling temperature threshold, increasing the charge current level of the device when the current temperature is below a falling temperature threshold for a first period of time, and decreasing the charge current level of the device when the current temperature is above the rising temperature threshold for the first period of time.

In various embodiments, the instructions, when executed by one or more processors, further cause the one or more processors to perform steps including determining the current temperature is above the rising temperature threshold for a second period of time and setting the charge current level of the device to a lowest charge current level.

In various embodiments, the instructions, when executed by one or more processors, further cause the one or more processors to perform steps including determining when the current temperature is above an alert threshold and immediately setting the charge current level of the device to a lowest charge current level.

In various embodiments, the instructions, when executed by one or more processors, further cause the one or more processors to perform steps including determining the current temperature is below the falling temperature threshold for a second period of time and increasing the charge current level by one level.

In various embodiments, the power is used to charge a battery.

In various embodiments, the current temperature is selected from the group consisting of a battery temperature and a processor temperature.

Additional features and advantages of the disclosed method and apparatus are described in, and will be apparent from, the following Detailed Description and the Drawings. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

The description will be more fully understood with reference to the following figures, which are presented as exemplary aspects of the disclosure and should not be construed as a complete recitation of the scope of the disclosure, wherein:

FIG. 1 illustrates a block diagram of a device according to an example aspect of the present disclosure;

FIG. 2 illustrates a block diagram of a system and its heat sources according to an example aspect of the present disclosure;

FIGS. 3-8 illustrates examples of thresholds in a thermal control process according to example aspects of the present disclosure; and

FIG. 9 illustrates a state diagram of a thermal control process according to an example aspect of the present disclosure.

### DETAILED DESCRIPTION

Turning now to the drawings, techniques are disclosed for new and innovative systems and methods for the thermal management of devices, such as medical devices and other electronic devices. The thermal management of medical devices becoming increasingly important due to increasing computational power and control of the medical devices. The increase in computational power and functionality is typically associated with a higher power draw, and the power draw of an electronic device is related to the amount of heat generated by the electronic device. The electronic devices typically operate within a particular temperature threshold, so the heat generated by the devices needs to be managed in order to keep the electronic device within the operating temperature threshold.

In several embodiments, medical devices include an infusion pump and/or a rack-mounted system having multiple infusion pumps. Infusion pumps can include, but are not limited to, peristaltic pumps, syringe pumps, ambulatory pumps, and/or any other pump delivering medications to a patient. It should be appreciated that the device is, in various embodiments, any type of medical device or any other suitable device having a rechargeable battery or other electronic components.

A variety of areas can be managed to manage the thermal performance of a device, such as but not limited to component temperature limits, device surface temperatures, and associated accessory surface and component temperatures in aggregate systems such as racks. In a variety of embodiments, fans or other active cooling devices can be used to manage the thermal performance of a device. However, these active cooling devices increase the overall cost of the device and increase power demand for the device as the active cooling devices require power. This can be particularly problematic for battery-powered devices that already have a fixed amount of power for the device. Active cooling devices may also have life limitations that are shorter than the overall life of the medical device, requiring repair and/or premature replacement of the medical device.

The complexity of the thermal management is further compounded by differing thermal time constants of the various component and devices, particularly within multi-device systems such as rack-mounted systems. Multiple devices generating heat independently in multi-device scenarios can be running under different conditions. Each of the devices internal systems can generate heat independently (function control, mechanism control, charging, etc.), and the performance of each device should be controlled independently in order to optimize the performance of each device. Similarly, a device can include multiple components, and the power draw for any particular component within the device can be controlled independently in order to optimize the performance of each component within the device. In a variety of embodiments, the rack-mounted system contains a number of infusion pumps and it is desirable to independently control each infusion pump based on the performance and/or temperature of each infusion pump. In this way, the performance of each infusion pump can be improved in order to optimize the delivery of various treatments to multiple patients.

Systems and methods as described herein can utilize multiple control levels for both time and temperature parameters while utilizing “no action zones” to limit unnecessary changes due to momentary change in temperature to control the performance of a particular component, device, and/or system to maintain performance within a particular thermal threshold. The temperature can be monitored via several thermistors (or any other temperature sensing device) within the components, devices, and/or system. The temperature can be measured for the device itself (e.g. a chassis temperature), for any component, and/or for combination of components as appropriate.

A variety of systems and processes in accordance with aspects of the disclosure are described in more detail below.

### Devices and Systems

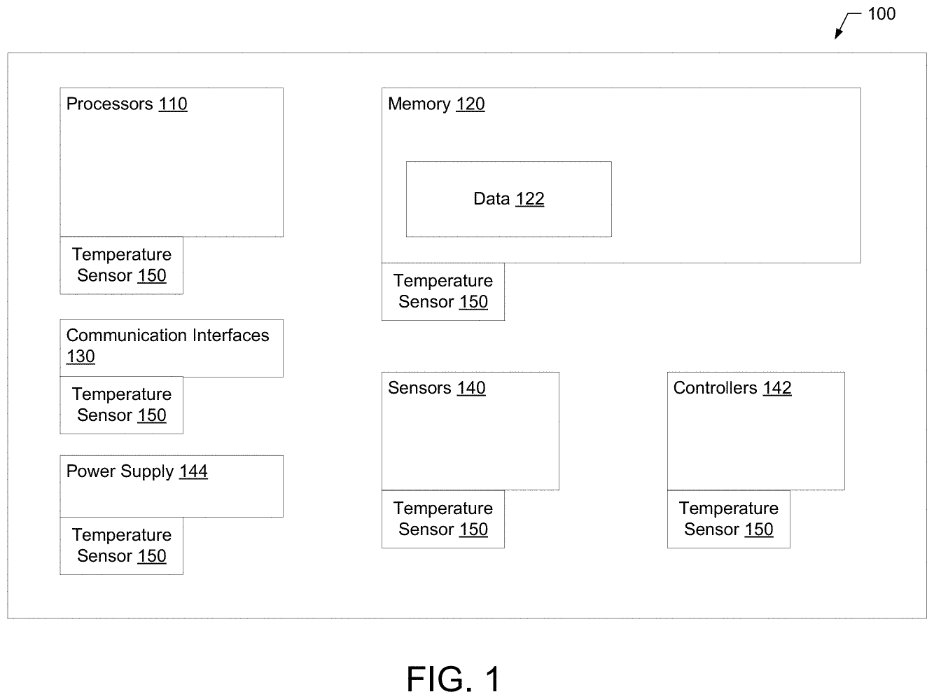

FIG. 1 illustrates a block diagram of a device according to an example aspect of the present disclosure. The device 100 can include processors 110, memory 120, communication interfaces 130, sensors 140, controllers 142, power supply 144, and/or temperature sensors 150.

The processor 110 may also be referred to as a central processing unit (CPU). The processor 110 can include one or more devices capable of executing instructions encoding arithmetic, logical, and/or I/O operations. In many aspects, the processor 110 may be a single core processor that is typically capable of executing one instruction at a time (or process a single pipeline of instructions) and/or a multi-core processor that may simultaneously execute multiple instructions. In a variety of aspects, the processor 110 may be implemented as a single integrated circuit, two or more integrated circuits, and/or may be a component of a multi-chip module in which individual microprocessor dies are included in a single integrated circuit package and hence share a single socket.

The memory 120 can include any combination of volatile and/or non-volatile memory devices, such as RAM, ROM, EEPROM, or any other device capable of storing data. In a number of embodiments, the memory 120 stores a variety of data 122. In a variety of embodiments, the data 122 causes the device 100 to provide one or more application programming interfaces (APIs) that provide data, such as temperature data and/or programming data, on request, on demand, automatically, on a schedule, or any other interval using the communication interfaces 130. In this way, the device 100 can report its thermal performance and/or obtain instructions to reconfigure its thermal management. Access to the APIs can be open and/or secured using any of a variety of techniques, such as by using client authorization keys, as appropriate to the requirements of specific applications of the disclosure.

Communication interfaces 130 can include a network device (e.g., a network adapter or any other component that connects a computer to a computer network), a peripheral component interconnect (PCI) device, storage devices, disk drives, sound or video adaptors, photo/video cameras, printer devices, keyboards, displays, etc. The communications interfaces 130 can communicate via a variety of networks as appropriate. These networks can include a LAN (local area network), a WAN (wide area network), telephone network (e.g. Public Switched Telephone Network (PSTN)), Session Initiation Protocol (SIP) network, wireless network, point-to-point network, star network, token ring network, hub network, wireless networks (including protocols such as EDGE, 3G, 4G LTE, Wi-Fi, 5G, WiMAX, and the like), the Internet, and the like. A variety of authorization and authentication techniques, such as username/password, Open Authorization (OAuth), Kerberos, SecureID, digital certificates, and more, may be used to secure the communications.

Sensor devices 140 can include a variety of sensors to sense a variety of environmental and/or physical conditions. For example, the sensor devices 140 can be used to measure and/or record data regarding a patient being treated for a particular condition. In another example, the sensor devices 140 can detect conditions of a room, such a temperature, humidity, light levels, and the like. Controllers 142 can include any devices used to perform actions. These actions can include, but are not limited to, adjusting an electrical output of a device, regulating the delivery of medicine, altering environmental conditions of a room, and the like. Power supply 144 can provide power to any of the components of device 100. The power supply 144 can include batteries, capacitors, transformers, charging circuity, and/or any other device capable of providing AC and/or DC power to the components of device 100. In a variety of embodiments, the power supply 144 includes an AC/DC converter that converts AC power into 3.3V, 5V, and/or 12V DC power to the components of device 100. Charging circuity of the power supply 144 can include any suitable charger, such as an AC charger, DC charger, solar panels, and the like.

The temperature sensors 150 can include any devices (e.g. thermistors) capable of measuring the thermal conditions of any of the components of the device 100. The temperature sensors can be embedded within particular components of the device 100 and/or separate temperature sensing devices. For example, the processor 110 may have an integrated thermal sensor, while a thermistor can be affixed to the memory 120.

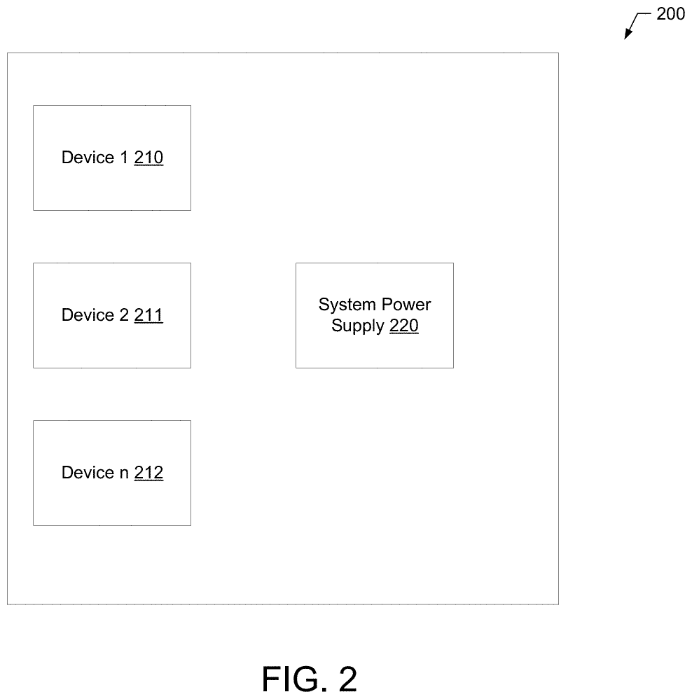

FIG. 2 illustrates a block diagram of a system and its heat sources according to an example aspect of the present disclosure. System 200 can include a variety of devices 210-212 and a system power supply 220. The devices 210-212 can be stored within a fixed space, such as a rack or other mounting system. The devices 210-212 can include any components of a device as described herein, such as with respect to FIG. 1. System power supply 220 can be a power device that provides power to one or more of the devices 210-212. The system power supply 220 can include AC power supplies, DC power supplies, and/or any other power supply as described herein.

### Thermal Control Processes

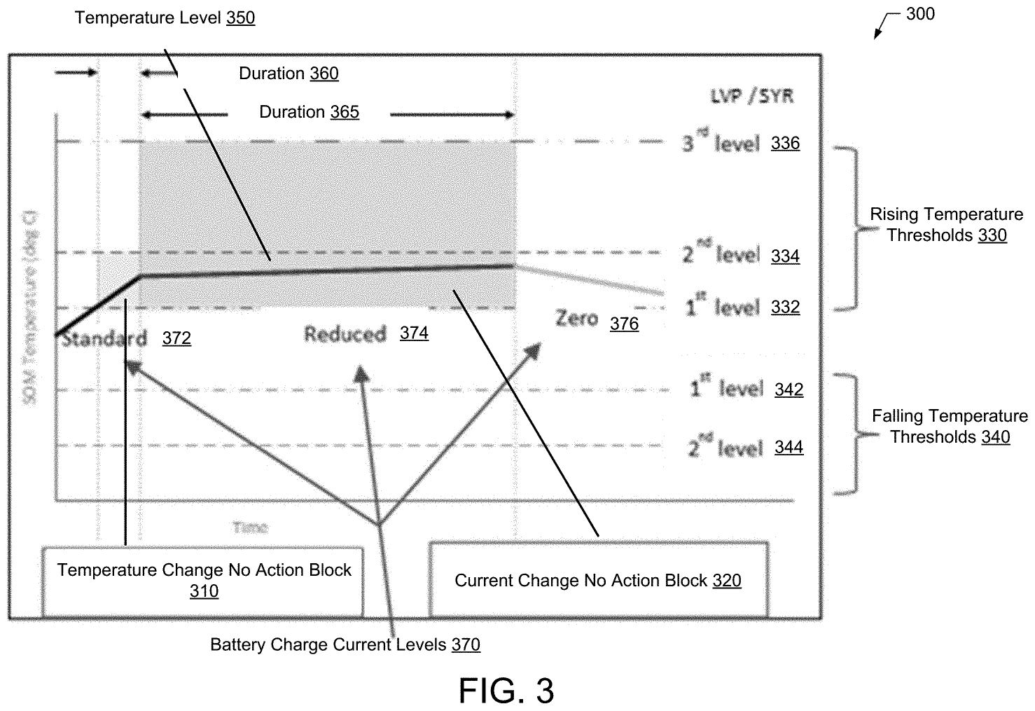

FIG. 3 illustrates an example of thresholds in a thermal control process according to an example aspect of the present disclosure. A variety of features of the thermal control process are shown in example 300, including three battery charge current levels 340. For example, battery charge current levels 370 can include a standard charge current 372, a reduced charge current 374, a zero charge current 376, and/or any other current level as appropriate to the requirements of particular applications of the invention. Example 300 includes three rising temperature thresholds 330, two falling temperature thresholds 340, a temperature change no action block 310 and a current change no action block 320. The temperature change no action block 310 can have a duration 360, such as 10 seconds as shown in example 300. During the temperature change no action block 310, the device operates at a standard battery charge current level 372, which causes the temperature 350 of the device to rise. Once the temperature 350 reaches a particular level and/or the duration 360 has elapsed, the battery charge current level 370 can be set to the reduced battery charge current level 374 and charging can continue at the reduced level in the current change no action block 320. The current change no action block 320 can have a duration 362, such as 150 second as shown in example 300. As shown in example 300, the temperature 350 continues to rise, but at a slower rate than in the temperature change no action block 310. Once the temperature 350 hits a threshold level and/or the duration 362 of the current change no action block 320 has elapsed, the battery charge current level 370 can be reduced to a zero charge current state 376. As shown in example 300, this causes the temperature 350 of the device to fall.

The component temperature can be divided into different ranges based on the various component, device, and system thresholds. As shown in example 300, the range of temperatures between the first rising temperature threshold 332 and the first falling temperature threshold 342 is the range where no changes to the component performance may be made, as these thresholds are associated with the temperature being too high to increase the performance (e.g. power) of the device, but it is also too low to decrease the performance (e.g. power) of the device.

The temperature range between the first rising temperature threshold 332 and second rising temperature threshold 334 corresponds to the range where the performance of the device will be reduced after waiting a threshold amount of time (e.g. temperature change no action block 310) to filter out any noise in the temperature reading. In this way, the temperature change no action block 310 can be used to filter noise in the temperature reading and prevent unwanted switching of the performance of the device (e.g. hysteresis). When the temperature 350 remains in this range after the hysteresis, the performance of the component, device, and/or system can be reduced.

The temperature range between the second rising temperature threshold 334 and the third rising temperature threshold 336 corresponds to the range where the performance will be immediately reduced by one level, without any hysteresis (e.g. an alert temperature threshold). In this range, this quick change is used to prevent the temperature 350 of the component, device, and/or system from rising any further. If the temperature 350 goes above the third rising temperature threshold 336, the performance is immediately reduced to zero. This is to reduce the thermal load within the component, device, and/or system as much as possible as the temperature can be approaching a level where the component, device, system may exceed its maximum operating temperature. Reducing the power to zero can help avoid damaging the component, device, and/or system and/or extend the effective lifespan of the component, device, and/or system.

The temperature range between the first falling temperature threshold 342 and second falling temperature threshold 344 can correspond to the range where the performance of the component, device, and/or system is increased by one level. If the temperature 350 goes below the second falling temperature threshold 342, the performance can be immediately increased by one level, even if the temperature change no action block 310 or current change no action block 320 is in effect. This can be done to increase the performance of the component, device, and/or system as soon as possible in order to take advantage of the low operating temperature.

Although example 300 is described with respect to battery charge current levels, any current level for any device or component can be utilized in accordance with aspects of the disclosure. Similarly, while only three charge levels, three rising temperature thresholds, and two falling temperature thresholds are shown in example 300, any number and/or granularity of charge levels and thresholds can be utilized depending on the specific requirements, such as a thermal time constant, of the system.

The following figures show the behavior of thermal control process using various examples of temperature to the changes in battery charge current. However, the same method applies to other components and devices as described herein.

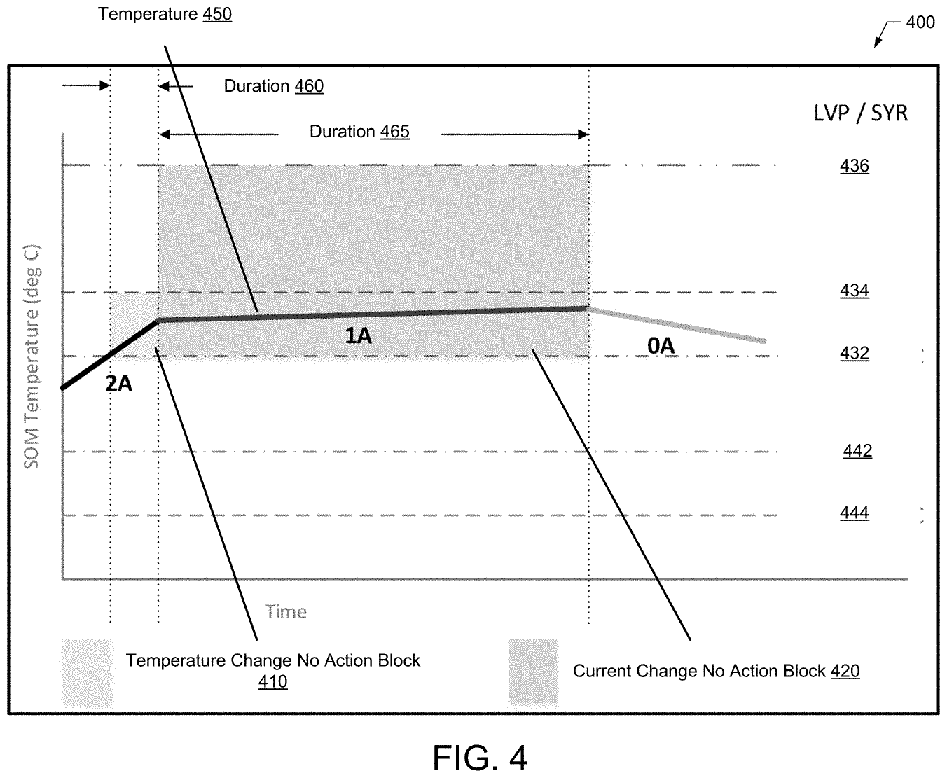

FIG. 4 illustrates an example of thresholds in a thermal control process when the temperature stays above first rising threshold according to an example aspect of the present disclosure. In example 400, the device is at an operating power of 2 amps. When the temperature 450 rises above the first rising temperature threshold 432, the temperature change no action block 410 is entered. This is a period of time (e.g. duration 460) after the temperature crosses a threshold during which any changes to the battery charge current are blocked in order to verify that the temperature 450 is going to remain beyond the threshold 432. Once the end of the temperature change no action block 410 is reached, and the temperature 450 is still above the threshold 432, the battery charge current is lowered by one level, for example to 1 amp as shown in example 400. Upon lowering the battery charge current, the current change no action block 420 is entered. This is a period of time (e.g. duration 465) after the battery charge current is changed during which any further changes to the battery charge current are blocked in order to allow the device to stabilize with the new battery charge current. If the temperature 450 is still above the first rising threshold 432 at the end of the current change no action block 420, the battery charge current can lowered again, this time to the lowest charge current level, for example 0 amps as shown in example 400.

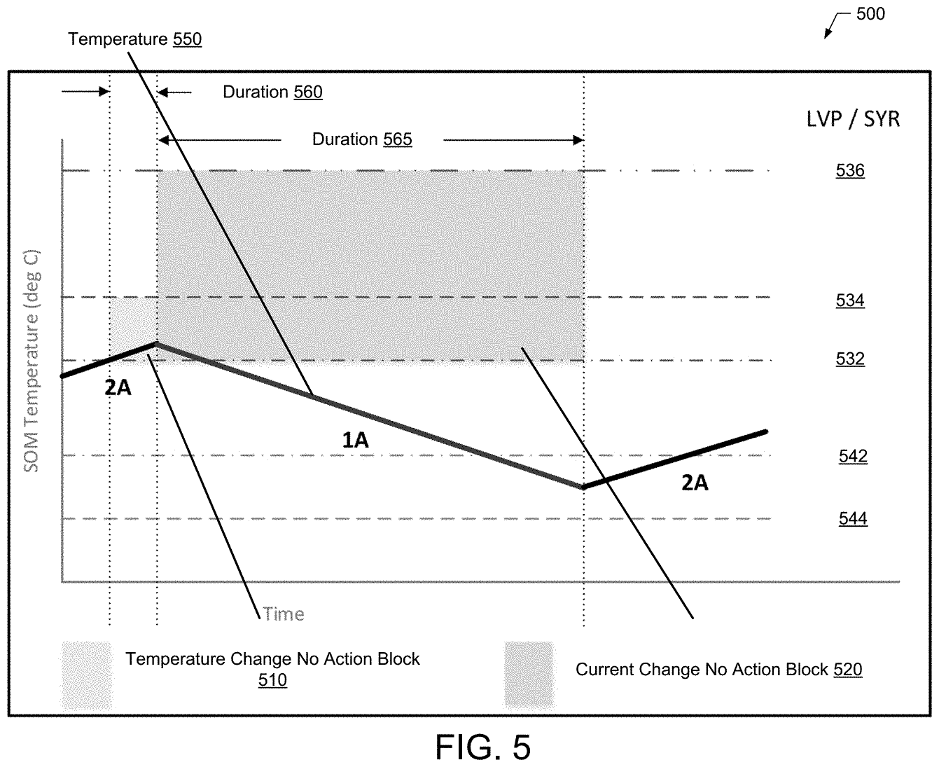

FIG. 5 illustrates an example of thresholds in a thermal control process when the temperature falls below first falling threshold according to an example aspect of the present disclosure. In example 500, the charge current of the device is 2 amps. When the temperature 550 rises above the first rising threshold 532, the temperature change no action block 510 is entered. Once the end of the temperature change no action block 510 is reached, the temperature is still above the threshold 532, so the charge current is lowered by one level (e.g. to 1 amp). Upon lowering the battery charge current, the current change no action block 520 is entered. In example 500, the temperature falls below the first falling threshold 542 at the end of the current change no action block 520. Accordingly, the battery charge current is increased by one level, or back to 2 amps.

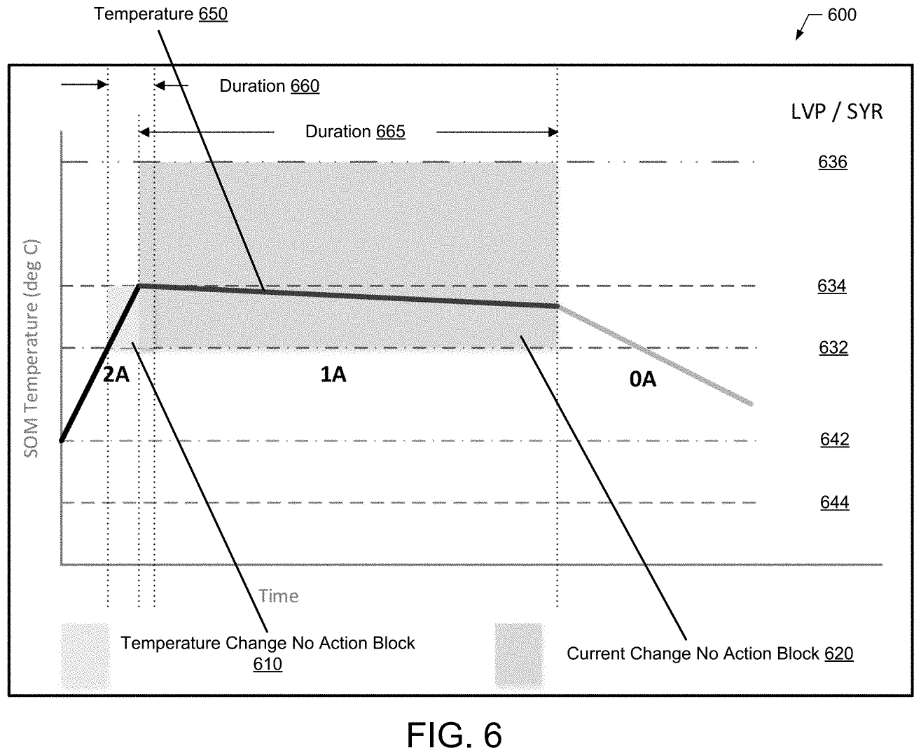

FIG. 6 illustrates an example of thresholds in a thermal control process when the temperature rises above second rising threshold according to an example aspect of the present disclosure. In example 600, the device is operating at 2 amps. When the temperature 650 rises above the first rising threshold 632, the temperature change no action block 610 is entered. In example 600, the temperature 650 continues to rise and crosses the second rising temperature threshold 634 before the end of the temperature change no action block 610 is reached. Therefore, the battery charge current is lowered by one level (e.g. to 1 amp) before the end of the temperature change no action block 610 is reached. After lowering the battery charge current, the temperature change no action block time threshold (e.g. duration 660) elapses and the current change no action block 620 is entered. The temperature 650 remains above the first rising threshold at the end of the current change no action block 620, therefore the battery charge current is lowered again, this time to the lowest charge current level, which is 0 amps in example 600.

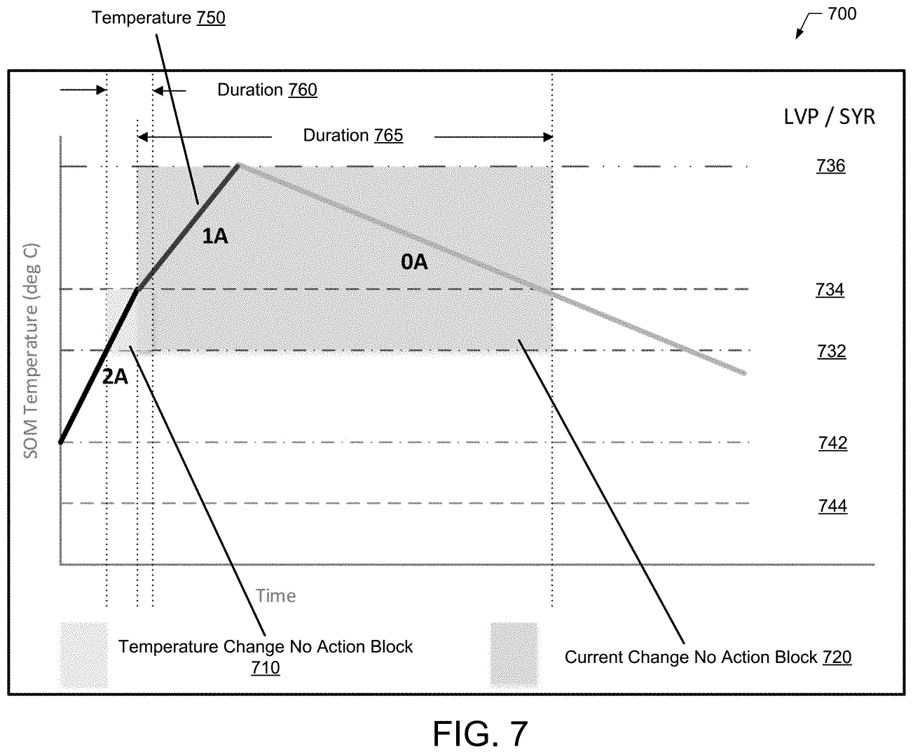

FIG. 7 illustrates an example of thresholds in a thermal control process when the temperature rises above third rising threshold according to an example aspect of the present disclosure. In example 700, the device is operating is operating at 2 amps. When the temperature 750 rises above the first rising threshold 732, the temperature change no action block 710 is entered. In example 700, the temperature 750 keeps rising and crosses the second rising temperature threshold 734 before the end of the temperature change no action block 710 is reached. Therefore, the current is lowered by one level (e.g. to 1 amp) before the end of the “temperature change no action block.” After lowering the battery charge current, the temperature change no action block time threshold (e.g. duration 760) elapses and the current change no action block 720 is entered. In example 700, the temperature 750 continues to rise and crosses above the third rising threshold 736 during the duration of the current charge no action block 720 (e.g. duration 765). At this point, the current is immediately lowered to the lowest charge level (e.g. 0 amps) without waiting until the end of the current change no action block 720. Once the current charge no action block 720 elapses, the temperature 750 remains above the first falling temperature threshold 742. Therefore, the current remains at the lowest charge current level.

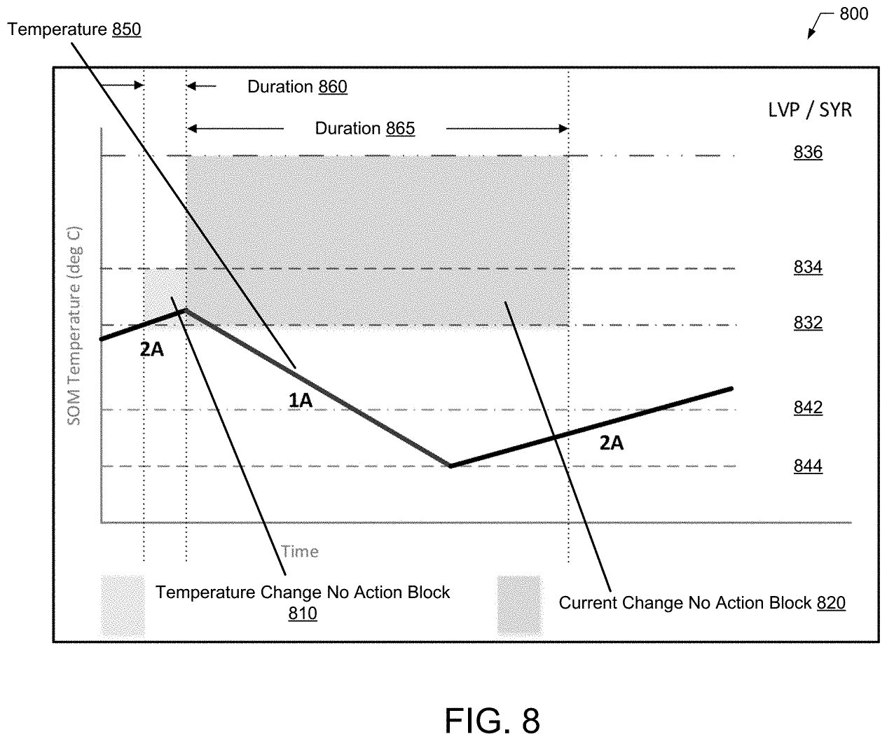

FIG. 8 illustrates an example of thresholds in a thermal control process when the temperature falls below second falling threshold according to an example aspect of the present disclosure. In example 800, the device is operating at 2 amps. When the temperature 850 rises above the first rising threshold 832, the temperature change no action block 810 is entered. Once the end of the temperature change no action block 810 is reached (e.g. after duration 860), the temperature 850 remains above the first rising threshold 832 and the battery charge current is lowered by one level, e.g. to 1 amp, and the current change no action block 820 is entered. In example 800, the temperature 850 falls below the second falling threshold 844 before the current change no action block 820 has elapsed (e.g. duration 865). Therefore, the battery charge current is increased by one level, to 2 amps, as soon as the second falling threshold 844 is crossed and before the end of the current change no action block 820.

Specific processes and examples for the thermal management of devices in accordance with embodiments of the invention are described with respect to FIGS. 3-8; however, any of variety of processes, including those that utilize different time periods, different temperature thresholds, and those for any arbitrary device, component, or system, can be utilized as appropriate to the requirements of specific applications of the disclosure. For example, a variety of different current states (e.g. 2.5 amps, 2 amps, 1.5 amps, 1 amp, 0.5 amps, and 0 amps) can be used to more finely control the power consumption of (and, relatedly, the temperature of) a particular device, component, or system. Similarly, a variety of different temperature thresholds (e.g. 65 C, 70 C, 75 C, 80 C, 85 C, 90 C, 95 C, 100 C, and the like) can be utilized.

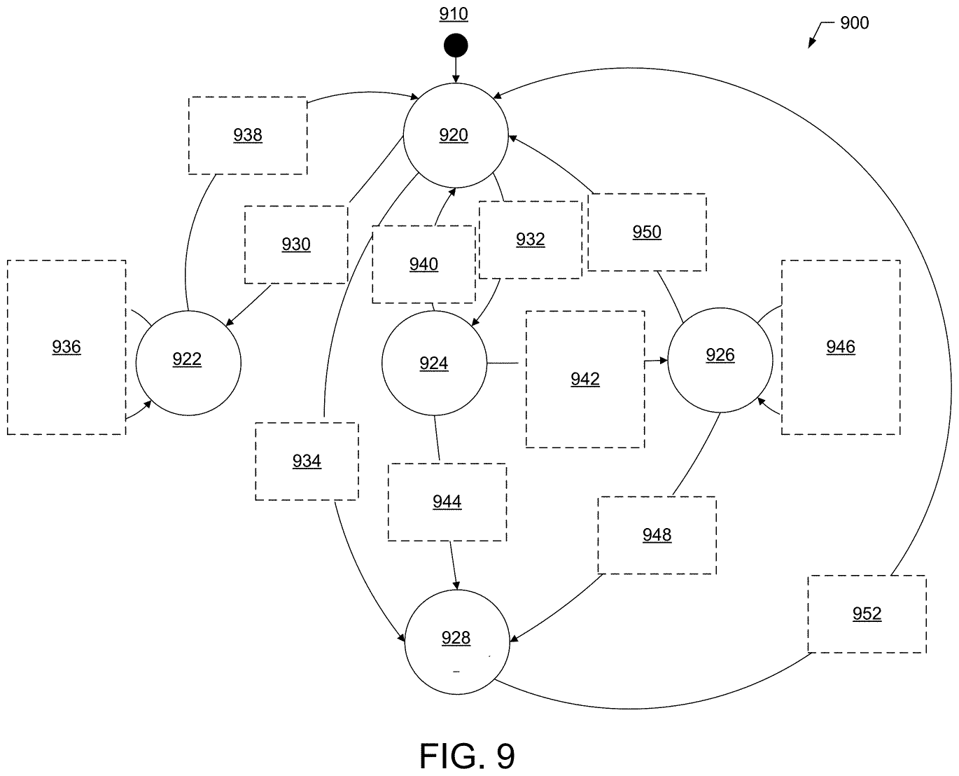

FIG. 9 illustrates a state diagram 900 of a thermal control process according to an example aspect of the present disclosure. The state diagram 900 begins at state 910, when the device is powered on. Once powered on, the device enters state 920, which corresponds to an initial operating state. While in state 920, when the device temperature drops below a falling temperature threshold, the device increases its charge current by one level and transitions 930 to state 922. When the device temperature exceeds a rising temperature threshold, the device transitions 932 to state 924. When the device temperature exceeds an alert temperature threshold, the device changes its charge current to the lowest level and transitions 934 to state 928.

State 922 corresponds to a current change no action block with the device temperature below a falling temperature threshold. In state 922, when the device temperature remains below the falling temperature threshold for a particular duration and/or falls below an immediate increase threshold, the device increases its charge current by one level (if not at the maximum charge current level) and remains 936 in state 922. When the duration elapses and the device temperature exceeds the falling temperature threshold, the device transitions 938 to state 920.

State 924 corresponds to a temperature change no action block. In state 924, when a duration has elapsed and the device temperature is below a rising temperature threshold, the device transitions 940 to state 920. When the device temperature remains above the rising temperature threshold and the charge current is above the lowest level, the device reduces its charge current by one level and transitions 942 to state 926. When the device is unable to reduce its charge current level, the device transitions 944 to state 928.

State 926 corresponds to a current change rising no action block. In state 926, when a duration has elapsed and the device temperature remains above the rising temperature threshold or when the device temperature exceeds an immediate decrease threshold, the device decreases its charge current by one level and remains 946 in state 926. When the device is unable to decrease its charge level, the device transitions 948 to state 928. When the duration has elapsed and the device temperature falls below the rising temperature threshold, the device transitions 950 to state 920.

State 928 corresponds to a charge current level at minimum with a high temperature block (e.g. an alert state). While in state 928, the charge level remains at its lowest state to allow the device to cool off. In state 928, when the device temperature falls below the temperature rising threshold, the device transitions 952 to state 920.

In a variety of embodiments, additional criteria regarding the environment in which the device exists can be used in the decision making of thermal control processes. For example, if the current charge of a battery is 25% of its capacity and the device is in high ambient temperature environment (e.g. the temperature of the environment is at or near the first, second, or third rising temperature threshold), the battery charging can be delayed until the device is located in an environment with a lower ambient temperature. In several embodiments, several reduced performance levels may be implemented. At a particular reduced performance level, the performance of the device may be reduced linearly with temperature if the system allows for it. For example, a battery can slowly reduce its charge rate as the battery's thermistor detects increasing temperature. In many embodiments, the temperature reading frequency (e.g. sampling rate) can be modified (e.g. increased and/or decreased) when approaching temperature limit to ensure that a sudden temperature spike is not missed.

It will be appreciated that many other methods of performing the acts associated with the state diagram 900 may be used. For example, the order of some of the states may be changed, certain states may be combined with other states, one or more states may be repeated, and/or some of the states described are optional. The state diagram 900 may be performed by processing logic that may include hardware (circuitry, dedicated logic, etc.), software, or a combination of both as described herein.

It will be appreciated that all of the disclosed methods and procedures described herein can be implemented using one or more computer programs, components, and/or program modules. These components may be provided as a series of computer instructions on any conventional computer readable medium or machine-readable medium, including volatile or non-volatile memory, such as RAM, ROM, flash memory, magnetic or optical disks, optical memory, or other storage media. The instructions may be provided as software or firmware and/or may be implemented in whole or in part in hardware components such as ASICs, FPGAs, DSPs or any other similar devices. The instructions may be configured to be executed by one or more processors, which when executing the series of computer instructions, performs or facilitates the performance of all or part of the disclosed methods and procedures. As will be appreciated by one of skill in the art, the functionality of the program modules may be combined or distributed as desired in various aspects of the disclosure.

Although the present disclosure has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present disclosure can be practiced otherwise than specifically described without departing from the scope and spirit of the present disclosure. Thus, embodiments of the present disclosure should be considered in all respects as illustrative and not restrictive. It will be evident to the annotator skilled in the art to freely combine several or all of the embodiments discussed here as deemed suitable for a specific application of the disclosure. Throughout this disclosure, terms like “advantageous”, “exemplary” or “preferred” indicate elements or dimensions which are particularly suitable (but not essential) to the disclosure or an embodiment thereof, and may be modified wherever deemed suitable by the skilled annotator, except where expressly required. Accordingly, the scope of the invention should be determined not by the embodiments illustrated, but by the appended claims and their equivalents.

What is claimed is:

1. A computer-implemented method, comprising:
measuring a current temperature of a device drawing power at a charge current level;
identifying a current temperature threshold in a plurality of temperature thresholds based on the current temperature, the plurality of temperature thresholds comprising at least one rising temperature threshold and at least one falling temperature threshold;
increasing the charge current level of the device when the current temperature is below a falling temperature threshold for a first period of time; and
decreasing the charge current level of the device when the current temperature is above the rising temperature threshold for the first period of time.

2. The computer-implemented method of claim 1, further comprising:
determining the current temperature is above the rising temperature threshold for a second period of time; and
setting the charge current level of the device to a lowest charge current level.

3. The computer-implemented method of claim 1, further comprising:
determining when the current temperature is above an alert threshold; and
immediately setting the charge current level of the device to a lowest charge current level.

4. The computer-implemented method of claim 1, further comprising:
determining the current temperature is below the falling temperature threshold for a second period of time; and
increasing the charge current level by one level.

5. The computer-implemented method of claim 1, wherein the power is used to charge a battery.

6. The computer-implemented method of claim 1, wherein the current temperature comprises a battery temperature.

7. The computer-implemented method of claim 1, wherein the current temperature comprises a processor temperature.

8. A device, comprising:
a processor;
at least one temperature sensor; and
a memory storing instructions that, when read by the processor, cause the device to:
measure, using the at least one temperature sensor, a current temperature of the device drawing power at a charge current level;
identify a current temperature threshold in a plurality of temperature thresholds based on the current temperature, the plurality of temperature thresholds comprising at least one rising temperature threshold and at least one falling temperature threshold;
increase the charge current level of the device when the current temperature is below a falling temperature threshold for a first period of time; and
decrease the charge current level of the device when the current temperature is above the rising temperature threshold for the first period of time.

9. The device of claim 8, wherein the instructions, when read by the processor, further cause the device to:
determine the current temperature is above the rising temperature threshold for a second period of time; and
set the charge current level of the device to a lowest charge current level.

10. The device of claim 8, wherein the instructions, when read by the processor, further cause the device to:
determine when the current temperature is above an alert threshold; and
immediately set the charge current level of the device to a lowest charge current level.

11. The device of claim 8, wherein the instructions, when read by the processor, further cause the device to:
determine the current temperature is below the falling temperature threshold for a second period of time; and
increase the charge current level by one level.

12. The device of claim 8, wherein the power is used to charge a battery.

13. The device of claim 8, wherein the current temperature comprises a battery temperature.

14. The device of claim 8, wherein the current temperature comprises a processor temperature.

15. A non-transitory computer readable medium storing instructions that, when executed by one or more processors, cause the one or more processors to perform steps comprising:
measuring a current temperature of a device drawing power at a charge current level;
identifying a current temperature threshold in a plurality of temperature thresholds based on the current temperature, the plurality of temperature thresholds comprising at least one rising temperature threshold and at least one falling temperature threshold;
increasing the charge current level of the device when the current temperature is below a falling temperature threshold for a first period of time; and
decreasing the charge current level of the device when the current temperature is above the rising temperature threshold for the first period of time.

16. The non-transitory computer readable medium of claim 15, wherein the instructions, when executed by one or more processors, further cause the one or more processors to perform steps comprising:
determining the current temperature is above the rising temperature threshold for a second period of time; and
setting the charge current level of the device to a lowest charge current level.

17. The non-transitory computer readable medium of claim 15, wherein the instructions, when executed by one or more processors, further cause the one or more processors to perform steps comprising:
determining when the current temperature is above an alert threshold; and
immediately setting the charge current level of the device to a lowest charge current level.

18. The non-transitory computer readable medium of claim 15, wherein the instructions, when executed by one or more processors, further cause the one or more processors to perform steps comprising:
determining the current temperature is below the falling temperature threshold for a second period of time; and
increasing the charge current level by one level.

19. The non-transitory computer readable medium of claim 15, wherein the power is used to charge a battery.

20. The non-transitory computer readable medium of claim 15, wherein the current temperature is selected from the group consisting of a battery temperature and a processor temperature.
